(19) United States
(12) Patent Application Publication
(10) Pub. No.: US 2003/0129739 A1
(43) Pub. Date: Jul. 10, 2003

(54) Analytical method and apparatus

(76) Inventor: Osborn Pierce Jones, Gwynedd (GB)

Correspondence Address: PAUL S MADAN, MADAN, MOSSMAN & SRIRAM, PC, 2603 AUGUSTA, SUITE 700, HOUSTON, TX, 77057-1130, US

(21) Appl. No.: 10/288,031

(22) Filed: Nov. 5, 2002

Related U.S. Application Data
(63) Continuation-in-part of application No. PCT/GB01/02045, filed on May 8, 2001.

(51) Int. Cl. C12M 1/34

(52) U.S. Cl. 435/287.2; 435/288.7; 435/288.6

(57) ABSTRACT

A method and apparatus for monitoring a microbial material in a fluid sample, the method includes, providing a fluid sample for microbiological analysis, and optionally selectively permitting multiplication of microbial material present in the fluid sample. The microbial sample is then permitted to enter a reaction chamber containing at least one capture member arranged to selectively capture the multiplied microbial material thereon and optionally washing the capture member having the microbial material captured thereon. The amount of the captured microbial material present on the capture member is subsequently monitored.

### CROSS REFERENCE TO RELATED APPLICATIONS

This application is a continuation-in-part of international application PCT/GB01/02045 filed on May 8, 2001 pursuant to the Patent Cooperation Treaty and designating the United States of America and which was an international filing of foreign priority application 0010910.8 filed on May 5, 2000 in the United Kingdom, each of which applications being incorporated herein by reference.

### BACKGROUND OF THE INVENTION

1. Field of the Invention

The present invention is concerned with an analytical method and apparatus for use in microbiological analysis or the like.

2. Description of the Related Art

Food-born illnesses represent a serious, recurring problem in almost all parts of the world. It is estimated that in 1998 some 2.2 million people, including 1.8 million children, died from contaminated food and water. The World Health Organisation (WHO) reports that 76 million cases of food borne diseases occur in the USA every year. This figure increases annually and now represents up to 30% of the population and results in 325,000 hospitalisation and 5000 deaths. In the USA alone diseases caused by major pathogens are estimated to cost up to US $37.1 billion.

Alarmingly, according to a recent edition of the WHO Statistics Quarterly surveys, foodborne diseases may be indicate. In Europe there is concern about the emergence of Salmonella, Campylobacter, Shigella, Listeria and Verotoxin Producing E Coli (VTECs) as major health threats.

Effective management of such diseases relies on a rapid identification of the particular pathogenic bacteria responsible for the disease. Analysis of the patient's faecal material remains the most direct method of determining the identity of such pathogenic bacteria. For instance, Campylobacter is not easily grown but due to the development of selective growth media many laboratories are able to screen for the organism.

While it is frequently possible to analyse samples of fluids such as body fluids or relatively clean liquids (ones not containing a great deal of solid material), it is frequently difficult to provide accurate biological analysis of “dirty” liquid samples, such as, for example, sewage slurry, abattoir waste, macerated food material, faeces or the like, all of which contain particulates, which can interfere with biological reactions.

At present microbiological analysis is carried out by the use of techniques such as growing bacteria on agar plates followed by visual identification of the resultant colonies. This method has a number of disadvantages including

Many bacteria take a significant time to grow or do so erratically under laboratory conditions e.g. Campylobacter, Brucella, Mycobacteria paratuberculosis

It is often difficult or impossible to differentiate between pathogenic and non-pathogenic strains e.g. VTECs from non pathogenic E Coli.

The cost, when labour is taken into consideration, is a significant factor.

It is therefore an object of the present invention to alleviate at least some of the problems indicated.

It is a further aim of the present invention to provide apparatus for the analysis of microbial material.

It is yet a further aim of the present invention to provide a method of monitoring microbial material in a fluid sample.

It is yet a further aim of the present invention to provide a method and apparatus of monitoring two or more microbial materials present in a fluid sample.

### SUMMARY OF THE INVENTION

According to the present invention, there is provided a method of monitoring a microbial material in a fluid sample, the method includes:

a) providing a fluid sample for microbiological analysis;

b) optionally selectively permitting multiplication of microbial material present in the fluid sample;

c) permitting the microbial sample to enter a reaction chamber containing at least one capture member arranged to selectively capture the multiplied microbial material thereon;

d) optionally washing the capture member having the microbial material captured thereon; and

e) monitoring the amount of the captured microbial material present on the capture member.

According to a further aspect of the present invention, there is provided a method of monitoring two or more microbial materials present in a fluid sample, the method includes:

a) providing a fluid sample for microbiological analysis;

b) optionally selectively permitting multiplication of microbial material present in the fluid sample;

c) permitting the microbial sample to enter two or more reaction chambers, each reaction chamber containing at least one capture member arranged to selectively capture the microbial material thereon;

d) optionally washing the capture member having the multiplied microbial material captured thereon; and

e) monitoring the amount of the captured microbial material present on the capture member.

The method according to the present invention is particularly suitable for the detection of verotoxin producing E coli (which are not easily identified, one from another on an agar plate), salmonella, campylobacter, mycobacterium paratuberculosis, shigella, yersinia, brucella, vibrio, aeromonas, listeria, clostridium difficile (toxin), verotoxins, giardia and criptospiridium. This list is exemplary and should not be considered as exhaustive.

The method optionally includes repeating step (d) one or more times prior to step (e). Washing the capture member has the advantage of removing and/or reducing the presence of background interfering bacteria, and also other interfering materials which may be present in the sample (for example solid debris and fats etc). Background interfering bacteria is considered to be any microbial material present in the sample which is not the specific microbial material being analysed. For example, if the present invention was being used to test the presence of E.Coli 0157, then other strains of E Coli would be considered to be a background interfering material.

Background interfering bacteria may also be reduced by the addition of antibiotics or the like, to the fluid sample prior to step (e) (this addition may be during step (b) or step (c), however during step (b) is preferred). The antibiotic is selected such that the microbial material being tested is not killed by the antibiotic whilst background interfering bacteria is killed.

It is particularly preferred that an enrichment broth is added to the fluid sample, typically during step (b). The enrichment broth typically includes nutrients so as to promote growth of the microbial material being tested but hinders the growth of background interfering bacteria. It is particularly preferred that the enrichment broth includes an antibiotic which, as discussed above, is capable of killing background interfering bacteria.

Preferably, each stage of the method is performed at a predetermined temperature. The predetermined temperature is typically in a range which promotes growth of the microbial material being tested (for example 37° C.).

Typically, the contents of the reaction chamber are agitated so as to mix the sample and the capture member. It is particularly preferred that the agitation occurs by alternating a source of vacuum and a pressure pulse on the contents of the reaction chamber. Advantageously, the agitation assists in the reaction between the capture member and the sample, thereby capturing the microbial material on the capture member.

According to a first embodiment of the present invention, the capture member may include an antibody coated substrate. The substrate typically includes magnetic beads (which is preferred), plastics beads, microdots, sponges, gauze, membranes, or the like. The substrate may also include a mesh or the like, typically of a plastics material. However, it is envisaged that the substrate may include any material which is capable of being coated with the antibody so as to selectively capture the microbial material. The substrate may also comprise an inner surface of the reaction chamber which is, in this particular embodiment, typically coated with the antibody. The substrate may be in a uniform or random array. The advantage of the substrate being in a uniform array is that it is possible to identify individual reactions which are taking place.

The monitoring of the captured microbial material, according to this particular embodiment, may include ELISA or ATP analysis. When ELISA is used it is preferred that an enzyme linked to a specific antibody is added to the sample prior to detection.

According to a second embodiment of the first aspect of the present invention, the capture member includes nucleic acid probes so as to capture thereon specific nucleic acid strands for the microbial material being tested. In this particular embodiment, the nucleic acid strands are multiplied utilizing PCR which is known in the art, where a definitive part of the bacterial chromosome is amplified. The multiplied nucleic acid strands are subsequently detected using nucleic acid hybridization technique(s).

In the case of very slow growing bacteria it is impractical to wait for a long time for the bacteria to multiply on its own. Therefore, in order to expedite the multiplication of specific DNA strands, PCR can be used. This also has the effect of being more sensitive as well as being faster. The PCR probes may be attached to the capture member. Using PCR alone does not establish whether the bacteria is alive or not and therefore, in some instances, where practical, an incubation stage may be used as an indicator of a live colony. The converse is also true, sub lethally injured bacteria may not culture but would multiply with PCR. Other bacteria just don't grow at all in lab conditions.

The multiplication of the microbial material typically occurs in an enrichment zone which is arranged intermediate the reaction chamber and a sample container containing the fluid sample. However, it is envisaged that the sample container may include the enrichment zone thereby eliminating the requirement of a separate vessel. It is also envisaged that the multiplication may occur in the reaction chamber. The enrichment zone is typically arranged to permit growth of micro-organisms present in the fluid sample.

The sample is typically diluted with, for example, buffered peptone water or the like, hepes buffer, an isotonic solution, or indeed any solution capable of diluting the sample without destroying the target bacteria, prior to selective multiplication. The dilution is carried out under aseptic conditions so as to prevent the introduction of additional bacteria which was not originally in the fluid sample entering the sample, and thereby preventing subsequent cross-contamination occurring. Dilution of the sample is advantageous as it makes the sample easier to handle. It also has the advantage of diluting out fat and the like. Advantageously, the sample may be diluted with, for example, a wetting agent which typically removes fat from the sample.

Preferably, the fluid sample is provided in a sample receptacle, the sample is typically drawn from the sample receptacle to the enrichment zone and/or the reaction chamber as a controlled volume. This is particularly desirable for quantitative analysis. Typically, the drawing of the controlled volume from the sample receptacle to the enrichment zone and from the sample receptacle to the reaction chamber is affected by vacuum suction. It is also envisaged that the microbiological material and the magnetic beads may be removed from the reaction chamber subsequent to step (e) by (for example) vacuum suction.

The method according to the invention can enable samples of liquid containing particulates to be subjected to analysis, without great interference by solids present in the sample.

Specifically, it is frequently possible according to the invention to draw repeated aliquots (of known volume) of the sample into the reaction chamber.

The method according to the invention is particularly useful for monitoring liquids containing microbial material such as members of the Enterobacteracea group of bacteria and in particular campylobacter, salmonella, shigella, or VTECs.

It is specifically possible in a preferred embodiment of the invention to provide individual aliquots of volume, typically of approximately 100 microlitre or less for analysis (this is significantly smaller than known techniques).

According to a further aspect of the present invention, there is provided apparatus for use in monitoring a microbial material present in a fluid sample, which apparatus includes:

at least one reaction chamber arranged to receive capture member for selectively capturing microbial material contained in the fluid sample; and

a device for monitoring microbial material captured on the capture member.

The capture member may be fixed in the reaction chamber or alternatively, the capture member are introduced to the chamber during use.

According to a first embodiment of the second aspect of the present invention, the capture member includes an antibody coated substrate. The substrate may include magnetic beads (which are preferred), plastics beads, microdots (which may be arranged on a solid substrate), sponges, gauze, membranes, or the like. The substrate may also include a mesh or the like, typically of a plastics material. It is envisaged that the beads, microdots and the like are arranged in a random or uniform array, depending upon the requirements of the system. The advantage associated with the arrangement being in a uniform array is that it is possible to identify individual reactions in an organized/uniform array. Alternatively, the capture member includes the inner surface of the reaction chamber which, in this particular embodiment, is coated with an antibody.

The antibody is typically specific to the microbial material being analysed. For example, if the apparatus is to be used for the analysis of E. coli 0157, the preferred antibody would be specific to that bacterium strain.

The magnetic beads are typically of ferromagnetic material, such as iron-filled polymer beads or the like. The shape of the or each reaction chamber is preferably in the form of a respective elongate tube.

According to a second aspect of this embodiment of the present invention, the capture member includes nucleic acid strand capture member. The capture member may be arranged on a substrate (which may be magnetic or plastics beads, microdots, sponges, gauze, mesh, membranes or the like). The substrate may be arranged in a random or uniform array. The capture member may be a DNA hybridization probe, which may, if desired, be arranged on a substrate as described above.

In this embodiment of the present invention, the detection includes the use of a detector suitable for use in nucleic acid hybridization technique. The detector may, for example, include DNA hybridization probes.

The apparatus typically includes an agitating device for agitating the contents of the reaction chamber. The agitation device may include the capture member (such as the magnetic beads), which in one embodiment of the present invention are substantially free to move within the reaction chamber. Alternatively, the agitation device includes a solenoid activated bar. However, it is preferred that the agitation device includes the interior configuration of the reaction chamber which preferably tapers from a first diameter portion to a second diameter portion in which the diameter of the second diameter portion is greater than the first diameter portion. In use, fluid entering the reaction chamber creates a turbulence as it passes from the first diameter portion to the second diameter portion, thereby agitating the contents of the reaction chamber. It is also envisaged that the agitation device may include a pump arranged to provide alternate pressure and vacuum.

The apparatus according to the invention typically includes a sample receptacle, such as a shaped container, a self-supporting sample tube, vial or the like. The sample receptacle is preferably selectively connectable to, and removable from, the reaction chamber and is optionally replaceable and/or disposable. When the receptacle is connectable in this manner, it is preferably shaped and dimensioned to engage with the enrichment zone (the latter being for diluting the sample drawn from the sample receptacle).

The apparatus preferably further includes a conduit or the like which is arranged to permit part of the sample to be transferred from the sample receptacle to the enrichment zone. Such a conduit preferably comprises an inlet tube in communication with a filter tube.

The reaction chamber is typically in the form of an elongate conduit having a first open end in communication with the enrichment zone, and a second open end.

The first open end is preferably arranged such that, in use, it is immersed in the fluid sample (that is, below the surface of the fluid sample). It is also preferred that the second open end, when in use, is substantially above the surface of the fluid sample. It is further preferred that the elongate conduit is substantially U-shaped; the trough of the U being substantially rounded, substantially pointed (for example V-shaped) or may be substantially flat.

It is especially preferred that the elongate conduit may have an undulating appearance. It is particularly preferred that the elongate conduit is sinusoidal in appearance. This is particularly preferred in the second embodiment. Advantageously, in the second embodiment the PCR may be carried out in the portion of the conduit at the peak and the detection is carried out in the portion of the conduit arranged at the trough.

The apparatus may also include a dilution zone provided with at least one entry port permitting entry of diluent into the apparatus. The entry port is preferably suitable for aseptic introduction of the diluent; for example, the port may include a penetrable, self-sealing elastomeric membrane or the like. The dilution zone is preferably arranged for communication of the sample to the or each reaction chamber. In a preferred embodiment of the second aspect of the present invention, the dilution zone and the enrichment zone are substantially the same zone.

The apparatus typically includes a volume control device arranged to draw a volume (which is typically predetermined) of microbial material typically from the enrichment zone and/or the sample receptacle) into the reaction chamber. The control device typically utilizes vacuum to draw the volume of the sample.

In a preferred embodiment, it is preferred that the reaction chamber includes a respective first end arranged to receive the sample and a second end arranged to receive the magnetic beads and, if relevant a washing substance. If desired the multiplied sample and the magnetic beads may subsequently exit the reaction chamber.

Preferably, the device for monitoring the captured microbial material includes a spectrometer for measurement of luminescence, fluorescence or absorbance of colour, a radioactivity measuring device or a microscope for examination of the magnetic beads. It is therefore preferred that the or each reaction chamber is translucent or transparent (for spectral analysis).

The apparatus preferably includes a vacuum source which, when in use, draws the sample into the reaction chamber, and subsequently, the sample and/or the magnetic beads out of the or each reaction chamber, if required.

The apparatus preferably includes a pressure source which, when in use, assists in the introduction of the coated substrate and the wash material into the reaction chamber. Advantageously, the vacuum services and the pressure source are arranged to work alternatively on the contents of the reaction chamber. This alternate working therefore, advantageously, provides mixing for the contents of the reaction chamber.

In a particularly preferred embodiment of the present invention, the enrichment zone has a greater internal volume than the or each reaction chamber. This feature has the advantage that it is possible to introduce repeated aliquots of the sample, having a known volume, into the or each reaction chamber.

It is preferred that the apparatus further includes one or more heaters, which are typically controlled heaters.

The apparatus typically further includes an overflow chamber arranged to receive sample which overflows from the sample container.

It is envisaged that the apparatus may be a self contained whereby substantially all of the components of the apparatus are connected in one system and/or sealed unit. The apparatus is typically of plastics material.

It is particularly preferred that the apparatus is disposable. Therefore, the apparatus is designed to be used only once so that each apparatus is discarded after use thereby obviating the need to clean the apparatus. In a particularly preferred embodiment of the present invention, the apparatus includes at least two reaction chambers and at least one sample receptacle. This embodiment is particularly advantageous when it is desired to monitor for the presence of at least two microbial materials being present in the same sample (the sample, of course, being contained within the sample receptacle). Preferably, each reaction chamber will therefore contain a different capture member.

The present invention further comprises an analytical kit comprising a plurality of apparatus according to the present invention, each apparatus according to the present invention being arranged to be located on a carousel. Each apparatus is preferably located on the carousel at a defined position, such that the carousel can be moved such that each apparatus is presented to a succession of injection, vacuum source, pressurized reagent dispensers and/or wash stations.

The kit preferably further includes drive device arranged to rotate the carousel. Preferably, the or each apparatus, when in use, is mounted about the periphery of the carousel.

Advantageously, the kit further includes a plurality of injection and/or analysis stations arranged adjacent the carousel, each station being capable of performing a separate method step. Each method step may include a method step according to the first aspect of the present invention.

It is particularly preferred that the kit is a sealed unit.

### BRIEF DESCRIPTION OF THE DRAWINGS

A preferred embodiment of the invention will now be described with reference to the accompanying drawings, in which:

FIG. 1 shows, schematically, a first stage in the method according to the invention;

FIG. 2 shows schematically a second stage in the method according to the invention;

FIG. 3 shows schematically a third stage in the method according to the invention;

FIG. 4 shows schematically a fourth stage in the method according to the invention;

FIG. 5 shows schematically a fifth stage in the method according to the invention;

FIG. 6 shows schematically as sixth stage in the method according to the invention;

FIG. 7 shows schematically as seventh stage in the method according to the invention;

FIG. 8 shows schematically as eighth stage in the method according to the invention;

FIG. 9 shows schematically a ninth stage in the method according to the invention; and

FIG. 10 shows schematically a tenth stage in the method according to the invention;

FIG. 11 represents a kit according to the present invention located on a carousel.

### DESCRIPTION OF THE PREFERRED EMBODIMENTS

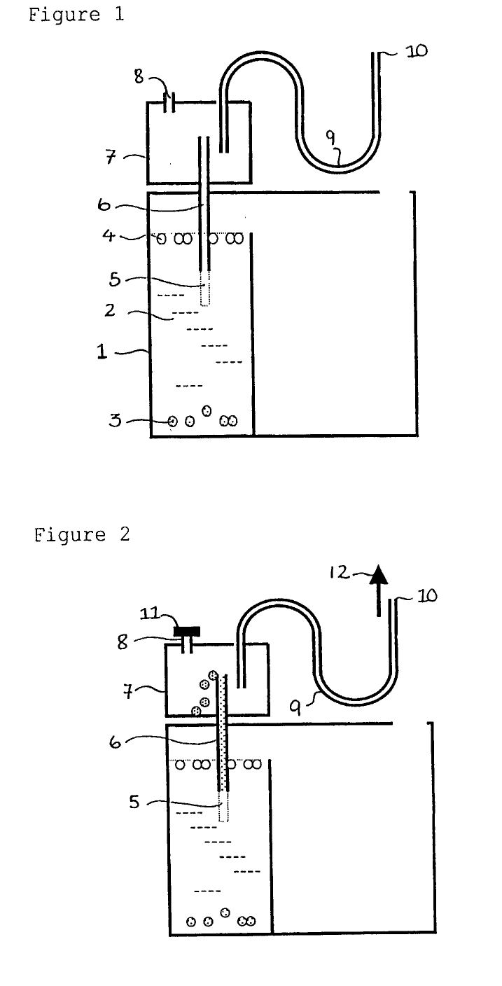

Referring to FIG. 1, there is shown a sample container 1 containing sample 2, the sample being primarily liquid but additionally containing sinking debris 3 and floating debris 4. A filter tube 5 connected to an inlet tube 6 permits communication of at least part of the sample to a selective enrichment chamber 7 which may contain capture beads for the removal of interfering substances.

Chamber 7 has an injection port 8 suitable for aseptic injection of enrichment or diluent broth or the like into the cell and to act as a vent.

A clear plastic conduit 9, has one end located in chamber 7 and the other end, 10, distally supported vertically in a position suitable for access by external probes and the like

Referring to the arrangement shown in FIG. 2, where like numerals have been used to indicate like parts shown in FIG. 1, a vacuum source 12 is connected via exit outlet 10 and port/vent 8, is temporarily sealed by sealing member 11. This means that an aliquote of sample can be drawn (utilizing vacuum source 12) from the sample container 1 into the enrichment chamber 7, as illustrated.

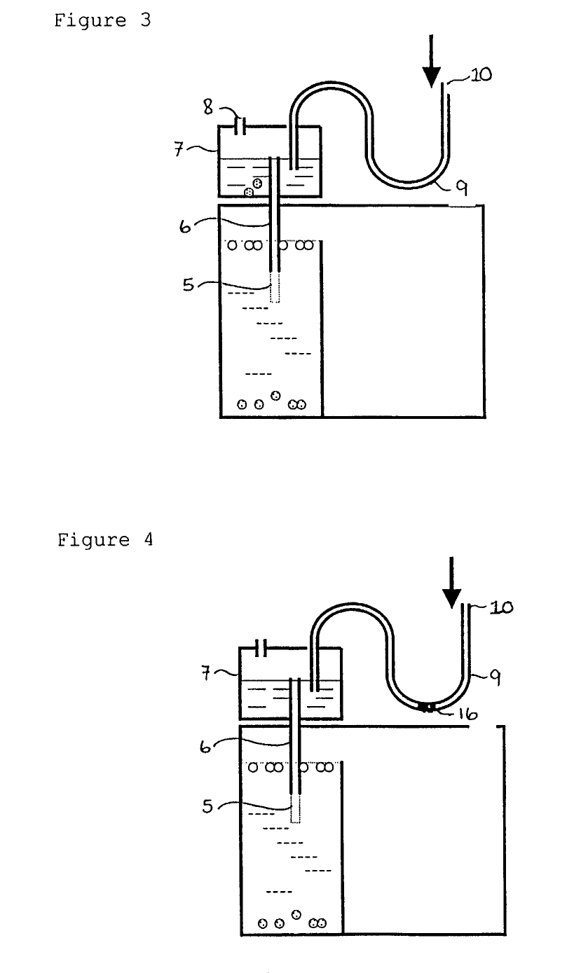

Referring to FIG. 3 where like numerals have been used to indicate like ports in the previous figures, Enrichment broth is introduced into enrichment chamber 7 through opening 10 in conduit 9, or through port 8.

Referring to FIG. 4, where like numerals have been used to indicate like ports in the previous figures, antibody coated magnetic beads, 16, can then be added via opening 10.

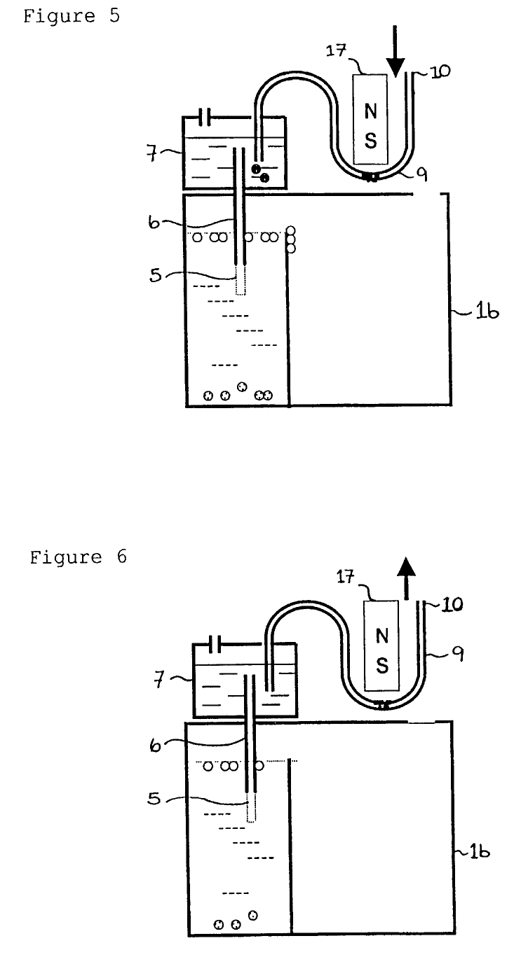

Referring to FIG. 5, where like numerals have been used to indicate like ports in the previous figures, the magnetic beads are concentrated using a permanent magnetic magnet 17 positioned in close proximity to conduit 9 which acts as a reaction chamber. An air stream is introduced through opening 10 in order to displace the bead suspension buffer into chamber 7. Any overflow is taken by overflow receptacle 1b.

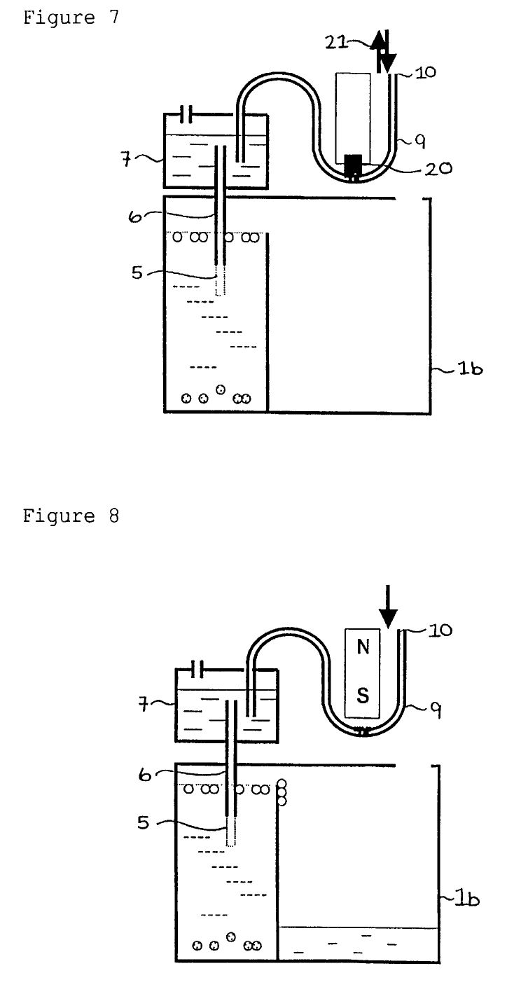

Referring to FIG. 6, where like numerals have been used to indicate like parts in the previous figures, a further vacuum is applied at port 10 with samples being drawn into the reaction chamber 9. At that point, with reference to FIG. 7, the magnetic beads and sample are mixed by the combined effect of solenoid activated bar 20, and an alternating source of vacuum and pressure pulse, 21, for re-suspension and reaction to take place.

In the reaction chamber, there is provided a liquid buffer, and magnetic carriers having immobilised thereon antibodies capable of immunologically bonding to a target substance. The buffer is preferably an aqueous liquid, such as phosphate-buffered saline or selective enrichment broth. Where appropriate, additional ingredients may be included such as antibiotics for reducing background interfering bacteria, for example.

The magnetic beads are typically of ferromagnetic material, such as iron-filled polymer beads or the like.

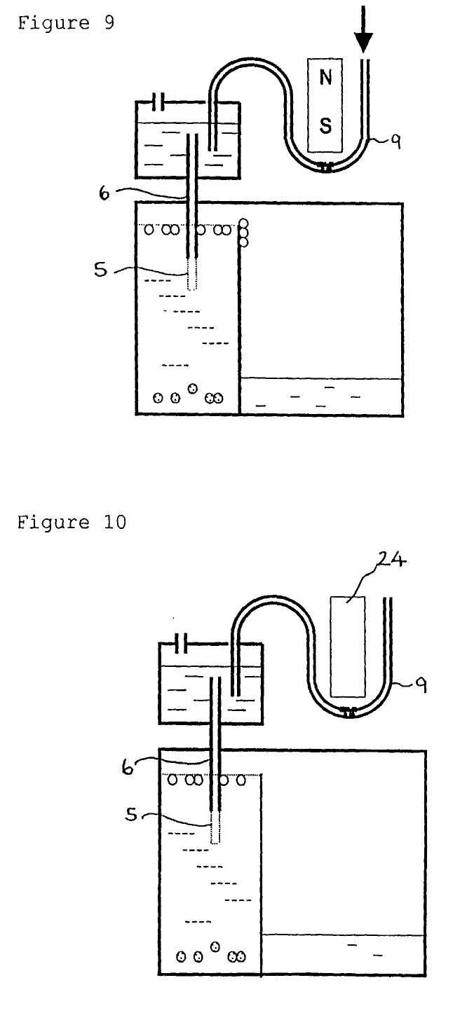

The contents of the reaction chamber may be agitated or trembled via electromagnetic means. The degree of immunological bonding to the magnetic beads may be monitored, typically by adding an aliquot of detection agent, 23, (FIG. 9) and measurement of luminescence, 24, (FIG. 10) radioactivity, fluorescence, absorbance of colour, microscopic examination of beads, the like.

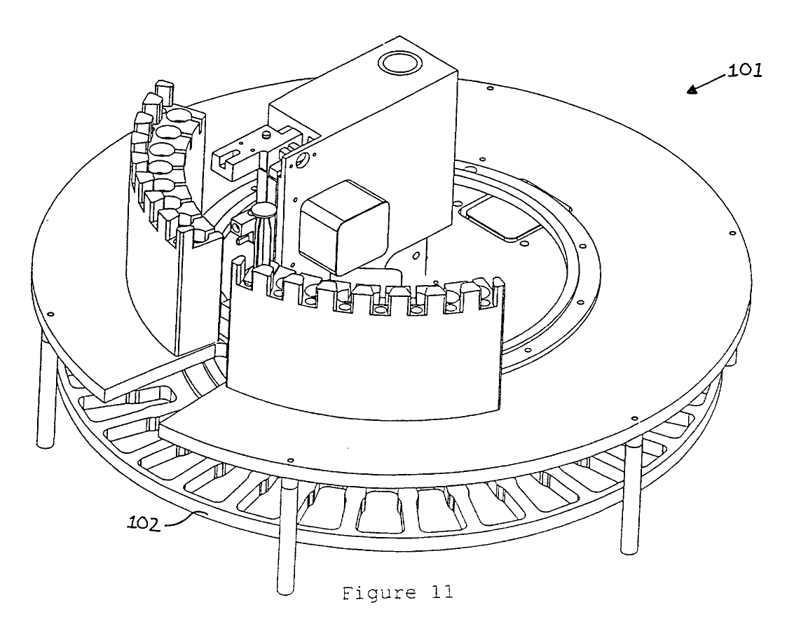

Referring to FIG. 11, there is provided a carousel generally indicated by the numeral 101. At least one apparatus according to the present invention is positioned in any of the slots in rotating tray 102. The tray 102 is moved such that each apparatus (not shown) is presented to a succession of injection and analysis stations.

What is claimed is:

1. An apparatus for use in monitoring a microbial material present in a fluid sample, which apparatus comprises:
at least one reaction chamber arranged to receive a capture member for selectively capturing microbial material contained in a fluid sample;
at least one enrichment zone arranged either (i) intermediate the reaction chamber and the sample container, or (ii) included in the sample container; and
a device for monitoring microbial material captured on the capture member.

2. An apparatus according to claim 1, wherein the capture member include an antibody coated substrate.

3. An apparatus according to claim 2, wherein the substrate includes magnetic beads, plastics beads, microdots (which may be arranged on a solid substrate), sponges, gauze, membranes, or the like.

4. An apparatus according to claim 3, wherein the magnetic beads are of ferromagnetic material, such as iron-filled polymer beads or the like

5. An apparatus according to claim 2, wherein the substrate is arranged in uniform or random array.

6. An apparatus according to claim 1, wherein the capture member includes the inner surface of the reaction chamber.

7. An apparatus according to claim 1, wherein the device for monitoring the captured microbial material includes a spectrometer for measurement of luminescence, fluorescence or absorbance of colour, a radioactivity measuring device or a microscope for examination of the magnetic beads.

8. An apparatus according to claim 1, wherein the capture member includes nucleic acid strand capture member.

9. An apparatus according to claim 8, wherein the capture member is selected from a group consisting of i) magnetic beads; ii) plastic beads; iii) microdots; iv) sponges; v) gauze; vi) mesh; and vii) membranes.

10. An apparatus according to claim 9, wherein the nucleic acid strand capture member includes a DNA hybridisation probe.

11. An apparatus according to claim 8, wherein the monitoring device includes a detector suitable for use in nucleic acid hybridization technique.

12. An apparatus according to claim 1, which further includes an agitating device for agitating the contents of the reaction chamber.

13. An apparatus according to claim 12, wherein the agitating device includes the capture member, a solenoid activated bar, or the interior configuration of the reaction chamber which preferably tapers from a first diameter portion to a second diameter portion in which the diameter of the second diameter portion is greater than the first diameter portion.

14. An apparatus according to claim 1, which includes a sample receptacle which is preferably selectively connectable to, and removable from, the reaction chamber.

15. An apparatus according to claim 1, which includes a conduit or the like which is arranged to permit part of the sample to be transferred from the sample receptacle to the enrichment zone.

16. An apparatus according to claim 1, wherein the reaction chamber is in the form of an elongate conduit having a first open end in communication with the enrichment zone, and a second open end.

17. An apparatus according to claim 16, wherein the first open end is arranged such that, in use, it is immersed in at least one of the fluid sample and the second open end, when in use, is substantially above the surface of the liquid fluid sample.

18. An apparatus according to claim 1, wherein the reaction chamber includes an elongate conduit which is preferably substantially U-shaped, the trough of the U being substantially rounded, substantially pointed or substantially flat.

19. An apparatus according to claim 1, wherein the reaction chamber includes an elongate conduit which has an undulating appearance.

20. An apparatus according to claim 1, which includes a dilution zone provided with at least one entry port permitting entry of diluent into the apparatus.

21. An apparatus according to claim 1, which includes a volume control device arranged to draw a predetermined volume of microbial material from at least one of i) the enrichment zone and ii) the sample receptacle into the reaction chamber.

22. An apparatus according to claim 1, wherein the reaction chamber includes a respective first end arranged to receive the sample and a second end arranged to receive the magnetic beads and, if relevant a washing substance.

23. An apparatus according to claim 1, which includes a vacuum source which, when in use, draws the sample into the reaction chamber, and subsequently, at least one of the sample and the magnetic beads out of the reaction chamber, if required.

24. An apparatus according to claim 1, which includes a pressure source which, when in use, assists in the introduction of the coated substrate and the wash material into the reaction chamber.

25. An apparatus according to claim 1, wherein the enrichment zone has a greater internal volume than the reaction chamber.

26. An apparatus according to claim 1, wherein the apparatus further includes a controllable heater.

27. An apparatus according to claim 1, wherein the sample container is arranged in an overflow chamber such that if the sample overflows the overflow is collected in the overflow chamber.

28. An apparatus according to claim 1, which is at least one of a self contained and a sealed unit.

29. An apparatus according to claim 1, which includes at least two reaction chambers and at least one sample receptacle.

30. An apparatus according to claim 29, wherein each reaction chamber contains a different capture member.

31. Apparatus according to claim 1, wherein the enrichment zone is arranged to permit growth of micro-organisms present in the fluid sample.

32. Apparatus according to claim 12, wherein the agitating device includes an alternating source of vacuum and pressure pulse arranged to provide alternate pressure and vacuum.

33. Apparatus according to claim 12, wherein the agitating device includes a pump.

34. Apparatus according to claim 1, which is disposable.

35. An analytical kit comprising a plurality of apparatus, each apparatus comprising:
at least one reaction chamber arranged to receive capture member for selectively capturing microbial material contained in a fluid sample;
at least one enrichment zone arranged either (i) intermediate the reaction chamber and the sample container, or (ii) included in the sample container; and
a device for monitoring microbial material captured on the capture member, each apparatus being arranged to be located on a carousel at a defined position, such that the carousel can be moved such that each apparatus is presented to one or more of a succession of injection, vacuum sources, pressurized reagent dispersers and wash stations.

36. A kit according to claim 35, which further includes drive device arranged to rotate the carousel.

37. A kit according to claim 36, wherein each apparatus, when in use, is mounted about the periphery of the carousel.

38. A kit according to claim 35, which includes at least one of a plurality of injection stations and analysis stations arranged adjacent the carousel, each station being capable of performing a separate method step.

39. A kit according to claim 35, which is a sealed unit.
